(19) United States
(12) Patent Application Publication
(10) Pub. No.: US 2011/0177198 A1
(43) Pub. Date: Jul. 21, 2011

(54) Isolated Microorganism Strain Lactobacillus Plantarum Tensia DSM 21380 as Antimicrobial and Antihypertensive Probiotic, Food Product and Composition Comprising Said Microorganism and Use of Said Microorganism for Preparation of Antihypertensive Medicine and Method for Suppressing Pathogens and Non-Starter Lactobacilli in Food Product

(75) Inventors: Epp Songisepp, Tartu (EE); Marika Mikelsaar, Tartu (EE); Merle Ratsep, Tartu (EE); Mihkel Zilmer, Tartu (EE); Pirje Hutt, Tartu (EE); Meeme Utt, Tartu (EE); Kersti Zilmer, Tartu (EE); Janne Uksti, Tartu (EE); Siiri Koljalg, Tartu (EE)

(73) Assignee: OU Tervisliku PIIMa Biotehnologiate Arenduskeskus (Biocompetence Centre of Healthy Diary Products), Tartu (EE)

(21) Appl. No.: 12/992,873

(22) PCT Filed: May 12, 2009
(86) PCT No.: PCT/EE2009/000005

§ 371 Date: Apr. 5, 2011

(30) Foreign Application Priority Data
May 13, 2008 (EE) P200800026

(51) Int. Cl. A23L 1/28; C12N 1/20

(52) U.S. Cl. 426/61; 435/252.9

(57) ABSTRACT

The present invention relates to a microorganism strain Lactobacillus plantarum Tensia DSM 21380 as antimicrobial and antihypertensive probiotic, food products (dairy products, e.g. fermented products or cheese) and composition comprising said microorganism and use of said microorganism for production of antihypertensive medicine. The invention also relates to a method for suppressing pathogens and nonstarter lactobacilli in food product and a method for extension of shelf life of food product by the means of Lactobacillus plantarum Tensia DSM 21380.

Isolated microorganism strain Lactobacillus plantarum Tensia DSM 21380 as antimicrobial and antihypertensive probiotic, food product and composition comprising said microorganism and use of said microorganism for preparation of antihypertensive medicine and method for suppressing pathogens and non-starter lactobacilli in food product

### TECHNICAL FIELD

The present invention relates to a novel microorganism and its applications. The invention belongs to the field of biotechnology and is used in food industry and medicine. More specifically the object of the present invention is antimicrobial and antihypertensive probiotic microorganism strain Lactobacillus plantarum Tensia DSM 21380. The invention relates to its use in the composition of a functional food e.g. cheese and for preparation of antihypertensive medicine.

### BACKGROUND ART

Lactobacilli used for production of fermented foods have been consumed for centuries. During the past decades lactobacilli have widely been used as probiotics. Probiotics are live microorganisms of human origin, which, when administered in adequate amounts, confer a health benefit on the host. Commonly probiotics are used for creating functional foods. Food can be regarded as functional, if beyond adequate nutritional components it contains some natural additives (pre- or probiotics), which beneficially affect one or more target functions in the body, either improving the state of health and well-being and/or reducing disease risk.

Probiotic products may be conventional foods (yoghurt, quark, curds) or dietary supplements (freeze-dried microbial cultures)

In European countries several probiotics supplied with health claims are marketed

### Antimicrobial Probiotics

Antimicrobial probiotics against opportunistic pathogens and food-borne pathogens e.g. Salmonella have been described in patent applications. In WO2008/039531 (Little Columet Holdings KKC) the application of strains of Lactobacillus acidophilus, L. bulgaricus, L. casei, L. paracasei, L. fermentum, L. plantarum, L. rhamnosus, L. salivarius, Bifidobacterium bifidum, B. infantis, B. animalis subsp. lactis, B. longum, Streptococcus thermophilus, Enterococcus faecalis and E. faecium is described. The oral administration of these strains helps to suppress Campylobacter jejuni, E. coli, S. aureus, Vibrio cholera, Bacteroids sp, Clostridium sp, Klebsiella sp, Listeria sp, Proteus sp, Salmonella sp, Shigella sp and Veillonella sp in gastrointestinal tract.

Patent application WO2008/016214 (Bioneer Corporation) describes a Lactobacillus gasseri strain BNR17 with antimicrobial activity against E. coli, S. aureus, S. typhimurium, B. cereus, L. monocytogenes and P. mirabilis.

Persons skilled in the art are aware of the problem caused in cheese by non starter lactobacilli (NSLAB), which appear during cheese ripening. These microbes can cause unwished proteolysis, appearance of various undesirable flavour compounds and loss of commercial quality of cheese.

Affecting the counts of NSLAB during cheese maturation process with pH regulators, antioxidants and preservatives (NaCl) has failed. The addition of different antimicrobial compounds producing (lactic acid and acetic acid, H₂O₂) antagonistic microbes incl. probiotic lactobacilli into cheese has also been ineffective. H₂O₂ producing probiotic lactobacilli have been described by several authors (Ouwehand, A. C. Westerlund, Antimicrobial components of lactic acid bacteria. In: Lactic acid bacteria: microbiological and functional aspects. Eds. Salminen, S., Wright, A., Ouwehand, A. C. 2004, pp 375-395, New York, Marcel Dekker; Hütt, P., Shchepetova, J., Lõivukene, K., Kullisaar, T., Mikelsaar, M. Antagonistic activity of probiotic lactobacilli and bifidobacteria against entero- and uropathogens. J. Appl. Microbiol., 2006, 100, 1324-1332).

Lactobacilli of human and plant origin adapt poorly in cheese environment rich in protein and fat and poor in carbohydrates, which is atypical for them. Therefore, only few probiotic lactobacilli comprising cheeses are available (Gardiner, G, Ross, R. P, Collins, J. H, Fitzgerald, G., and Stanton. C. Development of a probiotic cheddar cheese containing human derived Lactobacillus paracasei strains, Appl. Environ. Microbiol., 1998, 64, 6: 2192-2199; Ross, R P, Fitzgerald, G F, Collins, J K, O'sullivan, G C, Stanton, C G. Process for the manufacture of probiotic cheese, U.S. Pat. No. 6,872,411, Patent application WO 99/62348 AI), in which the added probiotic bacteria sustain their ability vegetable fermentation process (WO02/05665, WO0060948, Consejo Superior de Investigaciones Cientificas). The latter belongs to similar group of antimicrobial compounds with colicins, which attack mostly members of the same species and genus (Heinaru A, Tallmeister E. Colicin susceptibility of shigellas and coli bacteria related to episomal resistance and colicinogenicity or dissociation into S-R forms. Geneetika, 1971, 7, 5, 113-122 (in Russian). Therefore they are promising substances for suppression of undesirable nonstarter lactobacilli.

Production of plantaricins is regulated with several genes, some of which occur occasionally, some regularly. Therefore the phenotypical differences in plantaricin production occur. Genomic locus, associated with bacteriocin production has been described in Lactobacillus plantarum strain 11. Two of these operons of genes where termed plnEFI and plnJKLR, each having gene pair plnEF and plnJK encoding two small cationic bacteriocin-like peptides with double-glycine-type leader sequences. (Diep, D. B., Havarstein, L. S. & Nes, I. F. Characterization of the locus responsible for the bacteriocin production in Lactobacillus plantarum C11. J Bacterial 1996, 178, 4472-4483). No disclosure is available concerning L. plantarum strains that can suppress NSLAB and. possess full set of plantaricin encoding genes EF and JK.

### Antihypertensive Probiotics

More people suffer from metabolic syndrome which symptoms include overweight, obesity, and raise of blood pressure, blood glucose content and several other risk markers of atherosclerosis. Foods rich in fat (incl. cheese) are not problem-free, as these foods can increase the risk of developing atherosclerosis, inflammation, type II diabetes and/or lipid peroxidation (Ralf M., Tholstrup T., Basu S., Nonboe P., Sorensen M T, Straarup E M. 138. 509-514. A diet rich in conjugated linoleic acid and butter increases lipid peroxidation but does not affect atherosclerotic, inflammatory, or diabetic risk markers in healthy young men (American Society for Nutrition J. Nutr. 2008, 138:509-514). Conjugated linoleic acid (CLA) refers to two naturally occurring isomers of 18-carbohydrate linoleic acid (LA, cis-9, cis-12-18:2). CLA forms in the process of natural biohydrogenisation and oxidation. CLA isomers are formed during biohydrogenation of linoleic acid in the rumen and also through conversion of vaccenic acid in the mammary gland. In optimal dosages the conjugated linoleic acid (CLA) isomers have high health amelioration potential: antimicrobial, antitumorigenic, antidiabetic, anti-obesity and anti-allergic. Antihypertensive effect has also been demonstrated by several research groups (Inoue K., Okada F., Ito R., Kato S., Sasaki S., Nakajima S., Uno A., Saijo Y., Sata F., Yoshimura Y., Kishi R. and Nakazawa H., Perfluorooctane sulfonate (PFOS) and related perfluorinated compounds in human maternal and cord blood samples: assessment of PFOS exposure in a susceptible population during pregnancy, Environ. Health Perspect. 112 (2004), pp. 1204-1207).

Supplementation of CLA in high amounts with food is, however, problematic. It has been demonstrated, that consumption of 5 g CLA daily leads to the increase of lipid peroxidation (LPO), expressed in the elevated concentration (83%) of 8-isoprostaglandine F₂ (Raff M., Tholstrup T., Basu S., Nonboe P., Sorensen M T, Straarup E M. 138.509-514. A diet rich in conjugated linoleic acid and butter increases lipid peroxidation but does not affect atherosclerotic, inflammatory, or diabetic risk markers in healthy young men American Society for Nutrition J. Nutr. 2008, 138:509-514).

Best solution is the usage of lactobacillus strain possessing both the ability of moderate CLA production and physiologically relevant antioxidative capacity. Incorporation of physiologically relevant antioxidative LAB into food could control the increase of LPO and elevates the bio-suitability of fat-rich foods e.g. cheese. Lactobacillus fermentum ME-3 described in Tartu University patent EE04580 and in research articles possesses remarkable physiological antioxidative activity (Kullisaar T, Zilmer M, Mikelsaar M, Vihalemm T, Annuk H, Kairane C, Kilk A. Two antioxidative lactobacilli strains as promising probiotics. Int. Food Microbiol., 2002, 72, 215-224; Kullisaar, T., Songisepp, E., Mikelsaar, M., Zilmer, K., Vihalemm, T., Zilmer, M. Antioxidative probiotic fermented goats' milk decreases oxidative stress-mediated atherogenicity in human subjects. Br. J. Nutr., 2003, 90, 449-456; Truusalu, K. Naaber, P., Kullisaar, T., Tamm, H., Mikelsaar, R-H., Zilmer, K., Rehema, A., Zilmer, M., Mikelsaar, M. The influence of antibacterial and antioxidative probiotic lactobacilli on gut mucosa in a mouse model of Salmonella infection. Microbial Ecology in Health and Disease 2004, 16:4, 180-187).

Concentration of polyamines in human blood and exudation with urine fluctuate according to the consumption of food with different polyamines concentrations, but also in connection with endogenous polyamines producers in human intestinal microflora like E. coli and certain anaerobes (Marino M. Maifreni M, Moret S., Rondinini G. The capacity of Enterobacteriaceae sp. to produce biogenic amines in cheese. Letters of Microbiology, 2000, 31, 169-173). Several food products contain polyamines in relatively high concentrations e.g. oranges contain considerable amounts of putrescine 1330 μg/100 g (Larqué Sabater-Molina M., Zamora S. Biological significance of dietary polyamines. Nutrition, 2007, 23, 87-95.). Lactobacilli strains, which are able to produce moderate concentrations of CLA and polyamines and at the same time possess physiologically relevant antioxidativity have not been described.

Blood pressure is regulated by compounds like nitrogen monooxide (NO), lipid peroxidation (LPO), oxidized low density lipoproteins (ox-LDL) and components of glutathione red-ox system (GSSG/GSH), which modulate blood vessel stiffness and affect their vasoconstriction.

Moreover, the raise of LPO, ox-LDL, GSSG/GSH levels is one risk factor of atherosclerosis. Metabolism of acetylated spermidine provides additional possibilities for vasodilatory (blood pressure lowering) effect through acetylated form and polyamin ordinary forms (Myung C S, Blankenship J W, Meerdink D J. A mechanism of vasodilatory action of polyamines and acetylpolyamines: possible involvement of their Ca2+ antagonistic properties, J Pharm Pharmacol. 2000, 52:695-707).

Some peptides, harboured in milk possess blood pressure lowering ability through inhibition of angiotensine converting enzyme I (ACE) (Meisel, H. & Bocklemann, W. Bioactive peptides encrypted in milk proteins: proteolytic activation and tropho-functional properties. In: Proceedings of the sixth symposium on lactic acid bacteria: genetics, metabolism and applications. 19-23 September. Veldhoven (W. N. Konings, O. P. Kuipers, and J. Huis in't Veld., eds.) Kluver Academic Publishers, the Netherlands. 1999, pp. 207-215 (1999)).

Nitric monoxide NO belongs to bioactive compounds, having several beneficial effects like antimicrobial and anti-inflammatory activity and blood pressure lowering property, (Janeway, C A, Travers, P, Walport, M, Shlomchik, M J. 2005. Immunobiology: The Immune System in Health and Disease. New York, N.Y.: Garland Science Publishing).

NO affects blood pressure though different pathways like relaxation of smooth muscles of blood vessels, ACE inhibition or promotion of endogenous protection mechanisms (preconditioning) (Jones S P, Bolli R The ubiquitous role of nitric oxide in cardioprotection. J Mol Cell Cardiol 2006; 40: 16-23).

Today only a few disclosures are available concerning probiotic microorganisms, which produce NO endogenously or promote NO endogenous production. Lactic acid bacteria with promote endogenous NO production are described in patent applications of Valio Ltd Nos EE00200230A and EE200200231. Patent application EE00200230A discloses lactobacillus strain L. helveticus LBK-16H, DSM 13137 with photolytic activity and which produces di- and tri-peptides. Those di- and tri-peptides derived form casein due to lactic fermentation are concentrated by nano-filtration and when applied into food products incl. fermented milk products, an antihypertensive peptides containing food has been created. Patent application EE200200231 discloses, that strain L. helveticus LBK-16H, DSM 13137 in addition to di- and tri-peptide production ability is also able to induce NO production in two cell lines (mice and human enterocytes). The strain assists blood pressure lowering through the production of peptides and NO. The authors note, that the bacterial cells were not the only NO producers (page 13, lines 8-9).

U.S. Pat. No. 7,183,108 (Compagnie Gervais Danone, 2007) discloses, that Lactobacillus casei possesses anti-inflammatory ability due to the increase of NO production (NO is produced by proinflammatory cytokine activated enterocytes), and vice versa—the bacterial NO production is down regulated when enterocytes are already activated by pro-inflammatory cytokines and bacterial lipopolysaccharides.

Korhonen et al. 2001 (Korhonen K, Reijonen T M, Remes K, Malmström K, Klaukka T, Korppi M. Reasons for and costs of hospitalization for paediatric asthma: A prospective 1-year follow-up in a population-based setting, Pediatr Allergy Immunol 2001:12:331-338) showed, that Lactobacillus rhamnosus GG could stimulate NO production by intestinal epithelial cells or by induction of proinflammatory cytocines. These authors pointed out the possible desirable connection of LOG and NO production.

European patent EP0951290 (Laboratories Standa S.A., 2002) discloses NO-producing propionibacteria applied into the composition for production of NO in gastrointestinal tract, presumably in enterocytes. Ibidem a strain of Lactobacillus farciminis is mentioned as a negative example (the producer of low NO amounts). U.S. Pat. No. 7,294,337 (Institut National de la Recherche Agronomique, 2007) however demonstrates that L. farciminis is able to produce NO in amounts sufficient for anti-inflammatory effect and suppression of pain after peritoneum stretch.

It has been disclosed that L. fermentum strain LF1 was able to produce NO aerobically in MRS broth without the presence of human cell culture. The L. plantarum strain DSM9843 (LP2), tested by same authors, was not able to act similarly, opposite to our strain L. plantarum Tensia DSM 21380 (J. Xu, W. Verstraete. Evaluation of nitric oxide production by lactobacilli. Appl. Microbiol. Biotechnol., 2001, 56:504-507). Incorporation into human organism a strain as a probiotic with aforementioned properties allows controlling the blood pressure due to the NO production ability of the strain and the property of NO to affect blood pressure pathways, as described above.

Besides the poor survival in food products, the selection of probiotic bacteria faces the problem of poor survival of strains thought the passage of the gastrointestinal environment. Therefore, such probiotic lactobacilli are needed, which are able to survive both in the upper as well as the lower parts of gastrointestinal tract. The international patent application WO 91/05850 (Tartu University et al., 1989) describes the strain L. plantarum 38, as beneficial for adjusting colonic microflora in the case of bacterial dysbiosis. European patent EP0554418 B1 (Probi AB, 1998) discloses the good colonization properties of the strain L. plantarum 299 (DSM 6595) in the human intestine and the application of the strain with oatmeal-drink for surgical patients against enteric infection. International patent application WO2007/003917 A1 (Matforsk A S et al, 2006) describes Lactobacillus plantarum DSM 16997 (DSM 17320) strains and a strain of L. pentosus (DSM17321) as starter microbes in meat products. According to authors, these strains can function as probiotics balancing human intestinal microflora through suppression of harmful bacteria and also commensial Escherichia coli. To prove these claims, the results of in vitro experiments are presented. However, no proof of pathogen suppression abilities in vivo (i.e. on animal model) is given. Besides, the suppression of non-starter lactobacilli (NSLAB) is not demonstrated, although survival of NSLAB in sausage environment is described at low pH values. Without proof the probiotic strains are attributed immunostimulatory and sepsis-lowering properties after surgical interventions and the improvement of patients' well-being through the administration of aforementioned probiotic strains.

Patent EE04097 B1 (Probi AB, 2003) discloses the application of Lactobacillus plantarum 299v (DSM 9843) for treatment of urinary infections, whereas the strain is able to adhere to the human colonic epithelium because of its mannose-specific adhesins and to compete with harmful urinary tract pathogens for adhesion sites. According to REA (restriction enzyme type) analysis all aforementioned strains differ from each other and from Lactobacillus plantarum Tensia DSM. 21380 for the clearly distinct chromosome profile.

### DISCLOSURE OF THE INVENTION

The present invention relates to a novel isolated microorganism strain Lactobacillus plantarum Tensia DSM 21380 as antimicrobial and antihypertensive probiotic, food products (dairy products, e.g. fermented products or cheese) and composition comprising said microorganism and use of said microorganism for production of antihypertensive medicine. The invention also relates to method for suppressing pathogens and non-starter lactobacilli in food product and method for extension of shelf life of food product by the means of Lactobacillus plantarum Tensia DSM 21380.

Lactobacillus plantarum Tensia DSM 21380 as antimicrobial probiotic produces antimicrobial substances—lactic acid, acetic acid, CLA, NO, H₂O₂ and possesses plantaricin II b class gene pairs EF and JK encoding antimicrobial compound. With its metabolites Lactobacillus plantarum DSM 21380 suppresses in vitro both non-starter lactobacilli and enteric pathogens. The strain is able to germinate and predominate among lactobacilli at low temperatures and in the condition of carbohydrate starvation (e.g. cheese).

In addition to antimicrobial activity Lactobacillus plantarum Tensia DSM 21380 is also an antihypertensive and antioxidative probiotic due to the production of NO, CLA and antioxidativity, produces in vitro small amounts of polyamines tyramine and putrescine and prevails in the intestinal microflora of volunteers after 3-week consumption of Lactobacillus plantarum Tensia DSM 21380 comprising cheese. The good colonization ability of Lactobacillus plantarum Tensia DSM 21380 in the human intestine was detectable in fecal samples both by DGGE profile and by methods of classical bacteriology. Lactobacillus plantarum Tensia DSM 21380 significantly activates the metabolism of putrescine and acetylated spermidine in the human body, accompanied by lowering both systolic and diastolic blood pressure.

### DESCRIPTION OF THE STRAIN

The object of the invention, the microorganism strain Lactobacillus plantarum Tensia DSM 21380 was isolated from a fecal sample of a healthy child during a comparative study of the microflora of Estonian and Swedish children. The microorganism L plantarum Tensia DSM 21380 was isolated, by seeding the dilutions of the faeces of a healthy one-year old Estonian child (10⁻²-10⁻⁴ in phosphate buffer with 0.04% thioglycol acid; pH 7.2). The dilutions were seeded on freshly prepared MRS agar medium (Oxoid) and cultivated at 37° C. microearobically. The strain, which is the object of the invention, was isolated according to the characteristic morphology of colonies and cell to Lactobacillus sp. A provisional and more precise identification followed, as described below.

The strain, which is the object of the investigation, was isolated according to the characteristic morphology of colonies and cell to Lactobacillus sp. A provisional and more precise identification followed as described below.

The fact that the microbial strain Lactobacillus plantarum Tensia DSM 21380 originates from the intestinal tract of a healthy child, proves its GRAS (generally recognized as safe) status i.e. that this strain of microorganism is harmless for human organism and is suitable for oral application.

The microorganism Lactobacillus plantarum strain Tensia was deposited in Deutsche Sammlung von Mikroorganismen and Zellkulturen GmbH under the registration number DSM 21380 at 16.04.2008.

Cultural-morphological characteristics were detected after cultivation on MRS agar and in MRS broth (OXOID). Cells of Lactobacillus plantarum Tensia DSM 21380 are Gram-positive, non-spore-forming rods of regular shape, occurring singly, in pairs or in short chains.

### Physiological-Biochemical Characteristics

The MRS broth was suitable for cultivation of the strain during 24-48 h in microaerobic environment, after which homogenous turbid growth occurred in the broth. Colonies on MRS agar plates after 48 h of growth at 37° C. in microaerobic conditions (atmosphere CO₂/O₂/N₂: 10/5/85) are round, 2-2.5 mm of diameter, sooth, entire, convex and white.

The optimal growth temperature is 37° C.; it multiplies also at 15° C. and 45° C. The optimal pH of the growth environment is 6.5.

Lactobacillus plantarum strain Tensia DSM 21380 is catalase and oxidase negative, facultatively heterofermentative, no gas is produced from glucose and no arginine hydrolysis.

Strain Lactobacillus plantarum Tensia DSM 21380 was identified on the basis of biochemical activity with API 50CHL System (bioMérieux, France) kit as Lactobacillus plantarum (Coincidence with the type strain: Excellent, ID %-99.9, T index-0.86). The comparison with the reference strain Lactobacillus plantarum ATCC 14917 confirmed the preliminary identification by API 50CHL.

Carbohydrate utilization profile of Lactobacillus plantarum Tensia DSM 21380 according to API 50 CHL is as follows. Positive reaction for ribose, L-arabinose, galactose, D-glucose D-fructose, D-mannose, mannitol, α methyl-D-mannoside, a methyl-D-glucoside, N acetyl-glucosamine, amygdalin, arbutine, esculine, salitsin, cellobiose, maltose, lactose, melibiose, D-arabinose, sachharose, trehalose, melezitose, D-raffinose, β-gentiobiose, D-turanose, gluconate, weak reaction for starch.

According to API ZYM test-kit (bioMérieux, France), Lactobacillus plantarum Tensia DSM 21380 possesses leucine arylamidase, acid phosphatase, α-glucosidase, β-glucosidase and acetoin activity. Weak reaction for valine arylamidase, naphthol-AS-BI-phosphohydrolase, β-galactosidase, cystine arylamidase, esterase (C4), esterase (C8), N-acetyl-β-glucosaminidase was detected.

The molecular identification of Lactobacillus plantarum Tensia DSM 21380 was confirmed by Internal-Transcribed Spacer Polymerase Chain Reaction (ITS-PCR in comparison with the reference strain Lactobacillus plantarum ATCC 14917 (FIG. 1).

Method. Strain identification was confirmed by (ITS-PCR) Internal-Transcribed Spacer Polymerase Chain Reaction in comparison with the reference strain Lactobacillus plantarum ATCC 14917.

The DNA extraction from Lactobacillus isolates was performed using lysozyme (Serva, Sweden; 20 mg/ml), mutanolysin (Sigma; 0.5 mg/ml) and proteinase K solutions (Fermentas, Lithuania; 14.6 mg/ml).

The DNA amplification was performed in 1×Taq polymerase buffer, containing 1.5 U Taq polymerase (Fermentas, Lithuania), 0.5 μM of each primer (16S-1500F and 23S-32R; DNA Technology AS), 200 μM deoxynucleoside triphosphates (Amersham Pharmacia Biotech, Germany), 2 mM MgCl₂ and 2 μl of DNA under investigation.

Subsequently, the PCR product was restricted using a Taq I restriction enzyme (Fermentas, Lithuania). PCR products were separated by on a 2% agarose gel in 1×TBE buffer; voltage 100 V). The banding patterns were visualized in UV light and compared with reference stain L. plantarum ATCC 14917.

Lactobacillus plantarum Tensia DSM 21380 molecular fingerprints were compared with reference L. plantarum stain, using pulsfield-gel-electrophoresis (PFGE) (FIG. 2).

Method. For pulsfield-gel-electrophoresis (PFGE) procedure the lactobacillus strains were grown in MRS broth at 37° C. for 24 h. The cells were washed in SE buffer (75 mM NaCl, 25 mM EDTA, pH=7.4), density of the cell suspension was adjusted to 1.5 (OD₆₀₀). The DNA extraction from Lactobacillus isolates was performed in EC buffer (50 mM EDTA (pH 8.5), 0.5% Na-laurylsarcosine, 0.2%'Na-deocycholate 2 mg/ml lysozyme, 10 U mutanolysin), followed by 1 mg/ml proteinase K solution (100 mM EDTA-1% sarcosyl-0.2% deoxycholate, pH 8.0) containing buffer. Lysed probes were washed in TE buffer and cut to 2 mm, extracted overnight with enzyme 50 U Not I (Bio-Rad). The electrophoresis was carried out in CHEF-DR II (Bio-Rad) for 22 h at 14° C. The banding patterns were visualized in UV light illuminator.

### Antibiotic Resistance

Method. Lactobacillus plantarum Tensia DSM 21380 was tested using antibiotic strips of the E-test (AB Biodisk, Solna). The MIC (minimal inhibitory concentration) was determined according to European Commission (EUC) suggested epidemiological break-points.

TABLE 1
Antibiotic resistance of Lactobacillus plantarum Tensia DSM 21380

| | MIC* | MIC* | EUC antibiotic |
|---|---|---|---|
| | (μg/ml) | (μg/ml) | resistance |
| | L. plantarum | Control strain | MIC* |
| | Tensia | L. plantarum | breakpoint |
| Antibiotic | DSM 21380 | DSM 21379 | values (μg/ml) |
| Ampicillin | 0.25 | 0.19 | 4 |
| Gentamicin | 1.5 | 1 | 64 |
| Streptomycin | 16 | 6 | 64 |
| Erytromycin | 0.19 | 0.25 | 4 |
| Clindamycin | 0.032 | 0.016 | 2 |
| Tetracycline | 8 | 6 | 32 |
| Chloramphenicol | 2 | 2 | 8 |
| Cipro/ofloxacin | 32 | 32 | 4 |
| Quinopristin/dalfopristin | 1 | 1 | 4 |

*minimal inhibitory concentration

Lactobacillus plantarum Tensia DSM 21380 did not harbor antibiotic resistance against most important antimicrobial preparations.

Somewhat higher MIC for ciprofloxacin was detected in normal range of wild strains described previously. Therefore, likely it is wild type stain (Vankerckhoven V, Huys G, Vancanneyt M, Vael C, KlareI, Romond M-B, Entenza J M, Moreillon P, D. Wind R, Knol J, Wiertz E, Pot B., Vaughan E. E, Kahlmeter G, Goossens H. Biosafety assessment of probiotics used for human consumption: recommendations from the EU-PROSAFE project. Trends in Food Science & Technology 2008; 19: 102e114) and no horizontal transfer of antibiotic resistance genes of Lactobacillus plantarum Tensia DSM 21380 during the application of the strain as a probiotic could be predicted.

### Functional Properties

### The Profile of Metabolites

Method. The profile of metabolites was determined by gas chromatographic method (Hewlett-Packard 6890) after incubation in microaerobic milieu for 24 h and 48 h (Table 2). L. plantarum strain was grown on MRS agar for 48 h microaerobically (10% CO₂). A suspension (McFarland 4.0 turbidity standard, 10⁹CFU/ml) of lactobacillus culture was prepared in 0.9% NaCl solution. 1.0 ml of this suspension was transferred to 9.0 ml of MRS broth. The metabolite concentration (mmol/l) was detected using the capillary column HP-INNOWax (15 m×0.25 mm; 0.15 μm). The column temperature program was 60° C. 1 min, 20° C./min 120° C. 10 min; detector (FID) 350° C.

Electrochemical measurements of H₂O₂ were carried out with 24 h old intact cells in 500 μl of MRS broth with Apollo 4000 free radical analyzer (WPI, Berlin, Germany) and electrodes of type ISO-HPO2 and ISO-NOP.

ISO-HPO2 electrode signals were registered during 5-7 minutes. Mean signal strength was calculated. Each experimental point was measured in 4 independent parallels and each parallel was measured twice. H₂O₂ concentration was calculated according to the standard curves correlation with the strength of the electrodes signal.

TABLE 2
Acetic acid, lactic acid and succinic acid concentration
(mmol/l) of Lactobacillus plantarum Tensia DSM 21380
in MRS broth at microaerobic cultivation for 24 and 48 h and
H₂O₂ concentration (μg/ml) of intact cells

| | | | | | | | H₂O₂ |
|---|---|---|---|---|---|---|---|
| | Acetic acid | | Lactic acid | | Succinic acid | | (μg/ml) |
| | (mmol/l) | | (mmol/l) | | (mmol/l) | | intact cells |
| L. plantarum | 24 h | 48 h | 24 h | 48 h | 24 h | 48 h | 24 h |
| Tensia | 1.4 | 1.7 | 112.2 | 129.2 | 0.6 | 0.6 | 196.4 ± 128.8 |
| DSM 21380 | | | | | | | |
| DSM 21379 | 2.1 | 2.4 | 133.3 | 186.6 | 0.6 | 0.6 | 288.9 ± 175.8 |

Lactobacillus plantarum Tensia DSM 21380 produces substantial amounts of acetic acid, lactic acid and hydrogen peroxide. The concentration of the latter according to Apollo analyzer signals is however somewhat lower than that of the control strain.

### Antimicrobial Activity Against Pathogens and Non-Starter Lactobacilli

Lactobacillus plantarum Tensia DSM 21380 expresses in vitro on MRS agar medium antagonistic activity against non-starter lactobacilli and several enteric pathogens (Table 3). This property enables to use the strain for prolongation of shelf-life of food products.

TABLE 3
Lactobacillus plantarum Tensia DSM 21380 antimicrobial activity against
pathogens and non-starter lactobacilli on modified MRS agar medium
(pathogen growth inhibition zone, mm)

| Pathogen | Growth inhibition zone (mm) |
|---|---|
| Non-starter lactobacilli | 8.6 ± 4.07 |
| (NSLAB) | |
| Listeria monocytogenes | 25.1 ± 1.7 |
| Yersinia enterocolitica | 13.5 ± 1.7 |
| Salmonella enteritidis | 25.2 ± 1.5 |
| S. enterica serovar Typhimurium | 22.8 ± 0.1 |
| Shigella sonnei | 25.1 ± 1.6 |
| Escherichia coli | 29.8 ± 3.7 |
| Enterobacter sakazakii | 18.5 ± 3.61 |
| Campylobacter jejuni | 12.9 ± 5.2 |

Lactobacillus plantarum Tensia DSM 21380 antimicrobial activity in vitro in streak-line procedure (antimicrobial effect of metabolites) was highest against E. coli, followed by growth inhibition of Salmonella sp., Shigella sp and Listeria sp. The lowest antimicrobial activity of Lactobacillus plantarum Tensia DSM 21380 was against other lactobacilli (NSLAB).

Lactobacillus plantarum Tensia DSM 21380 Bacteriocin Genes

Method: Detection of Lactobacillus plantarum Tensia DSM 21380 bacteriocin encoding genes based on the Lactobacillus plantarum WCFS1 genes plnE, plnF plnJ and plnK.

These genes encode bacteriocin precursor peptides (Lactobacillus plantarum WCFS1 genome annotation—Kleerebezem et al 2003. Complete genome sequence of Lactobacillus plantarum WCFS1. Proc Natl Acad Sci USA. 2003 Feb. 18; 100 (4): 1990-5). The PCR primers were designed using Primer Express®. Lactobacillus plantarum WCFS1 annotated genome was downloaded from NCBI genome browser (http://www.ncbi.nlm.nih.gov/Genomes).

The designed primers were designated as E1F, Elk, F1F, F1R, J1F, J1R, K2F, K2R (look: Sequence listing) and there use in PCR-reaction gave positive signals for all aforementioned sequences. L. plantarum BAA-793 NCIMB 8826 was used as positive control and strain L. plantarum DSM 21379 as negative control.

TABLE 4
Presence of amplified PCR products of genomic DNA bacteriocin
encoding genes plnE, plnF, plnJ and plnK of Lactobacillus plantarum
DSM 21380

| | Genes | | | |
|---|---|---|---|---|
| | plnE | plnF | plnJ | plnK |
| L. plantarum BAA-793 (NCIMB 8826) | + | + | + | + |
| L. plantarum Tensia DSM 21380 | + | + | + | + |
| L. plantarum DSM 21379 | + | + | − | − |

The antagonistic activity of L. plantarum Tensia DSM 21380 among other antimicrobial compounds is also based on aforementioned gene products e.g. antimicrobial peptides. Peptides E & F and J & K should be expressed simultaneously to get max response via di-peptides.

Production of Conjugated Linoleic Acid (CLA) by L. plantarum Tensia DSM 21380

Method. Production of CLA was determined in MRS broth (de Mann-Rogosa-Sharpe, Oxoid, UK) and in skim milk by spectrophotometrical measurements.

| | | |
|---|---|---|
| MRS broth | 39.9 | mg/l (moderate) |
| Skim milk | 19.2 | mg/l (moderate) |
| Cheese | 3.0 ± 0.3 | mg/g |

Production of Nitrogen Mono-Oxide (NO) by L. plantarum Tensia DSM 21380

Method. Nitrogen mono-oxide production measurements were carried out with 24 h old intact cells in 500μ of MRS broth with Apollo 4000 free radical analyzer (WPI, Berlin, Germany) and electrodes of type. ISO-NOP electrode signals were registered during 5-7 minutes. Mean signal strength was calculated. Each experimental point was measured in 4 independent parallels and each parallel was measured twice. NO concentration was calculated according to the standard curves correlation with the strength of the electrodes signal.

TABLE 5
NO concentration (μM) produced by L. plantarum Tensia DSM 21380

| | NO |
|---|---|
| Strain number | concentrations (μM) |
| L. plantarum Tensia DSM 21380 | 2.6 ± 0.8 |
| L. plantarum DSM 21379 | 2.7 ± 1.2 |
| L. coprophilus | 2.1 ± 1.1 |
| L. plantarum | 2.1 ± 0.9 |
| L. paracasei ssp paracasei strain no1 | 1.3 ± 0.8 |
| L. paracasei ssp paracasei strain no 2 | 1.8 ± 0.9 |
| L. paracasei ssp paracasei strain no 3 | 2.8 ± 1.6 |
| L. buchneri | 2.0 ± 1.1 |

L. plantarum Tensia DSM 21380 was one of the best NO producers in comparison with 8 other Lactobacillus sp strains.

Antioxidative Activity of Lactobacillus plantarum Tensia DSM 21380

Method: For the detection of TAA and TAS of the microbial cells, the strain L. plantarum Tensia DSM 21380 was incubated in MRS broth (Oxoid, U.K.) for 24 h at 37° C. Microbial cells were harvested by centrifugation (1500 RPM, during 10 min) at 4° C. and the pellet was washed with isotonic saline (4° C.) and suspended in 1.15% KCl (Sigma, USA). The density of the suspension was OD₆₀₀ of 1.1×10⁹ bacterial cells ml⁻¹).

Total antioxidative activity (TAA) was assessed by using the linolenic acid test (LA-test). (Kullisaar, T, Songisepp, Mikelsaar M, Zilmer, K, Vihalemm, T, Zilmer, M. British J of Nutrition. Antioxidant probiotic fermented milk decreases oxidative stress-mediated atherogenicity in human. 2003, 90, 2, 449-456) and total antioxidative status (TAS) was measured by commercial kit (TAS, Randox Laboratories Ltd., UK) (Table 6).

TABLE 6
Total antioxidative activity (TAA) and total antioxidative status of (TAS)
Lactobacillus plantarum Tensia DSM 21380

| Strain | TAA (%) | TAS (mmol/l) |
|---|---|---|
| L. plantarum Tensia DSM | 22 ± 5 | 0.05 ± 0.02 |
| 21380 | | |
| L. plantarum DSM 21379 | 26 ± 1.2 | 0.13 ± 0.04 |

The values of TAA and TAS of the strain L. plantarum Tensia DSM 21380 were somewhat lower than of the strain. L plantarum DSM 21379.

L. plantarum Tensia DSM 21380 parameters were lower in comparison with the values of the antioxidative strain L. fermentum ME-3 (DSM14241) (ME-3, TAA: 29±0.7; TAS: 0.16±0.03).

In Vitro Polyamines Production of Lactobacillus plantarum Tensia DSM 21380

Method. Microbial strains were suspended in physiological saline according to McFarland turbidity standard (10⁹ CFU/ml) and 0.5 ml of each strain suspension was seeded into decarboxylation medium (a 4.5 ml) and incubated at 37° C. for 4 days (Bover-Cid et al., 1999).

For detection of BA 200 μl of medium was derivatized for GC analyze by modified method of Nakovichi (Nakovich, L. Analysis of biogenic amines by GC/FID and GC/MS. Thesis, Virginia polytechnic institute, USA. 2003).

GC analyses were carried out by gas chromatograph HP 6890 Series GC System, with capillary colonna HP-5 19091J-413 (30 m×0.32 mm; 0.25 μm). The column temperature program 160° C. 1 min., 20° C./min 280° C. 15 min; and detector (FID) 300° C.

TABLE 7
Concentration of polyamines in vitro in decarboxylation medium Arena, M. E., Manca de Nadra, M.
C. Biogenic amine production by Lactobacillus. J. Applied Microbiology 2001, 90, 158-162.

| | Polyamines (μg/ml) and biogenic amines | | | | | | | | |
|---|---|---|---|---|---|---|---|---|---|
| | Arginine | | Glutamine | | Lysine | | Ornithine | | Histidine |
| Sample | Putrescine | Cadaverine | Putrescine | Cadaverine | Putrescine | Cadaverine | Putrescine | Cadaverine | Histamine |
| L. plantarum | 0 | 0 | 0 | 0 | 0 | 0.3 | 0.5 | 0.6 | 0 |
| Tensia | | | | | | | | | |
| DSM 21380 | | | | | | | | | |
| L. plantarum | 0 | 0.4 | 1.2 | 0.5 | 0 | 0.4 | 1.9 | 0 | 0 |
| DSM 21379 | | | | | | | | | |
| E. coli | 18.4 | 1.7 | 12.3 | 18.4 | 1.8 | 240 | 1599.3 | 3.5 | 105.1 |
| ATCC 700336 | | | | | | | | | |

L. plantarum Tensia DSM 21380 produces small amounts of polyamine putrescine from ornithine. From biogenic amines, traces of cadaverine were detected. No production of histamine.

### DESCRIPTION OF THE DRAWINGS

FIG. 1. Lactobacillus plantarum Tensia DSM 21380 molecular identification by ITS-PCR
1. L plantarum DSM 21379
2. L. plantarum Tensia DSM 21380
3. L. plantarum CRL 972 (ATCC 14917)
M-100 bp marker (Fermentas)

FIG. 2. L. plantarum Tensia DSM 21380 molecular fingerprints in comparison with L. plantarum control strain (Pulse field-gel-electrophoresis profile, PFGE).
1. Lambda Ladder PFG Marker (New England Bin Labs Inc.)
2. L. plantarum Tensia DSM 21380
3. L. plantarum DSM 21379 (control)

FIG. 3. L plantarum Tensia DSM 21380 presence in cheese by DGGE method DGGE gel-electrophoresis form test-cheeses (cheeses with probiotic additive, control cheese, cheese with prebiotic additive).
1. Cheese nr 529
2. Control cheese
3. Cheese nr 5
4. L. plantarum strain no 1
5. Cheese nr 9-1
6. L. plantarum DSM 21379
7. Cheese nr 19
8. L. plantarum strain no 1
9. Cheese nr 9-2
10. L. plantarum DSM 21379
11. L. plantarum Tensia DSM 21380 cheese
12. L. plantarum Tensia DSM 21380 pure culture
13. Cheese nr 4
14. Cheese nr 12
15. L. paracasei strain no 1
16. L. plantarum DSM 21379

FIG. 4. Lactobacillus species by Pearson UPMAG cluster analyses in L. plantarum Tensia DSM 21380 group.

FIG. 5. Reduction of systolic blood pressure (end of the trial in comparison with the values of systolic blood pressure at the recruitment)

(a) Positive correlation with the minimal increase of lactobacilli counts (FISH) after the consumption of L. plantarum Tensia DSM 21380 comprising cheese (r=0.615, p=0.044, n=11).
(b) correlation with the increase of putrescine in urine of volunteers after the consumption of L. plantarum Tensia DSM 21380 comprising cheese in comparison with the values at the recruitment (r=0.631, p=0.037, n=11).

### DESCRIPTION OF THE EMBODIMENTS

### Example 1

### Suppression of NSLAB Microbes in Food Product

Test with Estonian Cheese

Method. Microorganism Lactobacillus plantarum Tensia DSM 21380 was added to the cheese milk of Dairy Cooperative E-Piim, (inoculation dose 3×10⁸CFU/vat) and the milk was renneted (25 min) The curds were cut (25 min), heated (34° C. 15 min), dried (25 min), pressed, drained (1 h), salted in brine (12° C.; 20% NaCl; pH 4,7) 20 h, drained and dried (8 h), backed into plastic and ripened at 12° C. for at least 4 weeks.

TABLE 8
Lactobacilli content in L. plantarum Tensia DSM 21380 comprising cheese at 28^{th} day of ripening.

| | | | L. plantarum Tensia DSM | | | | | |
|---|---|---|---|---|---|---|---|---|
| | L. plantarum | | 21380 comprising cheese | | | | | |
| | Tensia | Control | (used in volunteer trial) | | | Control cheese | | |
| | DSM21380 | cheese | Day | Day | 3 | Day | Day | 3 |
| | comprising cheese | pH 5.1 | 3. | 28. | months | 3. | 28. | months |
| Lactobacillus sp | 10⁹ | 9 × 10⁵ | 6 × 10⁵ | 5 × 10⁸ | 5 × 10⁷ | ND | 10⁸ | 10⁷ |
| L. plantarum | 10⁹ | 2 × 10³ | 6 × 10⁵ | 5 × 10⁸ | 5 × 10⁷ | ND | ND | ND |
| L. casei | ND | 8 × 10⁵ | ND | ND | ND | ND | ND | ND |
| L. buchneri | ND | 10⁵ | ND | ND | ND | ND | ND | ND |
| OHOL* | ND | ND | ND | ND | ND | ND | 2 × 10³ | ND |

OHOL—obligatively homofermentative lactobacilli
ND—not detected

The L. plantarum counts were 10 thousand times higher in L. plantarum Tensia DSM 21380 comprising cheese to the day 28 to in comparison with the control cheese. While in control cheese the homofermentative lactobacilli, L. casei and L. buchneri strains were present, then all aforementioned species were missing in L. plantarum Tensia DSM 21380 comprising cheese. Thus the strain Lactobacillus plantarum Tensia DSM 21380 possesses the ability to inhibit cheese NSLAB and suppress the cheese contaminating miorobiota. The latter can be pathogens occurring in food product after preparation. Therefore L. plantarum Tensia DSM 21380 addition could help to lengthen the shelf-life of a food product.

Detection of Lactobacillus plantarum Tensia DSM 21380 Counts in Cheese by DGGE

Method. Microbial DNA was isolated from cheese by QIAamp DNA Mini Kit (QIAGEN) and amplified with primers 968-GC-f (GGGAACGCGAAGAACCTTA-GC), 1401-r (CGGTGTGTACAAGACCC).

PCR products were separated by DGGE electrophoresis on a 30-60% acrylamide containing gel using Dcode™ System technique (Bio-Rad, Hercules, Calif.) (FIG. 3).

Detection of Biogenic Amines in L. plantarum Tensia DSM 21380 Comprising Cheese

Method. Cheese samples were extracted (20 ml 50% methanol solution was added to 10 g of cheese and incubated at 45° C. for 1 h, cooled to 30° C. and centrifuged) and 200 μl of upper layer was derivatized for GC analyze by modified method of Nakovichi (Nakovich, L. 2003 Analysis of biogenic amines by GC/FID and GC/MS) in Department of Microbiology of the University of Tartu.

GC analysis were carried out by gas chromatograph HP 6890 Series GC System, with capillary colonna HP-5 19091J-413 (30 m×0.32 mm; 0.25 μm). The column temperature program 160° C. 1 min, 20° C./min 280° C. 15 min; and detector (FID) 350° C.

TABLE 9
Biogenic amines and polyamines in L. plantarum Tensia DSM 21380 comprising
test-cheeses from industrial test trials

| | Viable counts of | | | | |
|---|---|---|---|---|---|
| | strain | | | | Viable count |
| | incorporated | | | | of |
| | into cheese | | | | L. plantarum |
| | (CFU/g) at day | | | | Tensia DSM |
| | 3-4 after | Amines (mg/kg) | | | 21380 in ripe |
| Sample | preparation | Tyramine | Putrescine | Cadaverine | cheese |
| L. plantarum | 6.5 × 10⁸ | 0.69 | 1.32 | 0 | 10⁶ |
| Tensia DSM | | | | | |
| 21380, 1. Batch | | | | | |
| Control cheese | — | 2.31 | 1.82 | 0 | — |
| 1. Batch | | | | | |
| L. plantarum | 4.5 × 10⁶ | 2.65 | 7.46 | 0 | 6 × 10⁸ |
| Tensia DSM | | | | | |
| 21380, 2. Batch | | | | | |
| Control cheese | | 5.64 | 1.84 | 0 | 10⁷ |
| 2. Batch | | | | | |
| L. plantarum | 2 × 10⁶ | 5.49 | 7.29 | 0 | 10⁸ |
| Tensia DSM | | | | | |
| 21380, 3. Batch | | | | | |
| Control cheese | — | 0 | 0 | 0 | — |
| 3. Batch | | | | | |

L. plantarum Tensia DSM 21380 produced tyramine 0.69-5.49 mg per kg of cheese and putrescine in lower quantities: 1.32-7.46 mg/kg.

### Example 2

### Lactobacillus plantarum Tensia DSM 21380 Safety Trial with NIH Mice

For the acceptance by the Estonian Veterinary and Food Board, the safety of probiotic strains and the food containing theses strains should be tested.

Method. In the experimental model with NIH mice 3 groups of mice consumed different cheeses during 30 days (control cheese with no additives, Lactobacillus plantarum Tensia DSM 21380 comprising cheese). Mice stayed in good condition, cheese administration caused increase of body weight, no changes in fur and digestion was detected. No translocation of administrated strains or other microbes into blood or organs was detected. No pathological shifts were found by the morphological and histological evaluation of the liver and spleen which proves the safety of Lactobacillus plantarum strain Tensia DSM 21380.

### Example 3

### Effect of Lactobacillus plantarum Tensia DSM 21380 Comprising Cheese on Blood Indices and Intestinal Microflora of Healthy Volunteers

The aim of the clinical trial (randomized blinded cross-over placebo controlled) was to evaluate the safety and effect on intestinal microflora of cheese comprising antimicrobial Lactobacillus plantarum Tensia DSM 21380 on healthy volunteers.

Persons and Methods: Participants were healthy volunteers, both male and female 12 persons (M/F 5/7; 2143 years). For exclusion criteria diabetes, glucose and glycohemoglobin HbA1c from blood sera were detected.

Test cheeses comprised strain Lactobacillus plantarum Tensia DSM 21380 (viable counts of 5×10⁸ CFU/g cheese). Before consumption the test-cheese was incubated with Lactobacillus plantarum DSM 21380 Tensia for 30 days at 12° C. Regular Estonian cheese without additives served as a control. Trial was a randomized blinded cross-over placebo controlled trial. Trial started with 3-week consumption of test cheese, followed by 2 week washout period, after which the control cheese was consumed for 3 weeks. Dose 50 g/day.

TABLE 10
Clinical data of healthy volunteers after consumption of probiotic cheese comprising
L. plantarum Tensia DSM 21380

| | Probiotic cheese | | | | |
|---|---|---|---|---|---|
| | with L. plantarum | | | | |
| | Tensia DSM 21380 | | Control cheese | | |
| | | After | | After | |
| | Baseline | treatment | Baseline | treatment | P values |
| BMI (kg/m²) | 24.1 ± 3.6 | 24.2 ± 3.6 | 23.8 ± 3.5 | 23.9 ± 3.6 | 0.625/0.399 |
| Systolic | 112.9 ± 10.4 | 107.1 ± 10.4 | 110.3 ± 8.3 | 109.3 ± 9.4 | 0.016/0.655 |
| pressure | | | | | |
| (mmHg) | | | | | |
| Diastolic | 78.8 ± 7.1 | 74.3 ± 8.8 | 74.8 ± 5.2 | 75.7 ± 8.3 | 0.021/0.668 |
| pressure | | | | | |
| (mmHg) | | | | | |

Body mass index (BMI) (kg/m²): 19-25 kg/m² - normal, 26-30 kg/m² - overweight, over 30 - obesity.

Both systolic and diastolic pressure were significantly lower after 3-week consumption of probiotic L. plantarum Tensia DSM 21380 (5×10⁸CFU/g×50 g) comprising cheese. The 3-week consumption of strain Tensia DSM 21380 comprising cheese did not increase the body mass index.

Effect of Lactobacillus plantarum Tensia DSM 21380 on Intestinal Microflora

Method. Quantitative analysis of fecal microflora and species of lactobacilli, methods elaborated in Department of Microbiology of Tartu University were (Mikelsaar M E, Väljaots M E, Lenzner A A. Anaerobe Inhalts-und Wandmikroflora des Magen-Darm-Kanals. Die Nahrung, 1984, 23, 6/7, 727-733; Sepp E., Julge K., Vasar M., Naaber P., Björksten B., Mikelsaar M. Intestinal microflora of Estonian and Swedish infants. Acta Paediatrica, 1997, 86, 956-961).

TABLE 11
Lactobacilli counts in the faeces of healthy volunteers (log₁₀ cfu/g faeces)

| | L. plantarum Tensia DSM | | | | |
|---|---|---|---|---|---|
| | 21380 comprising cheese | | Control cheese | | P values |
| | BL1 | PRO | BL2 | PL | paired t-test |
| | mean ± stdev | mean ± stdev | mean ± stdev | mean ± stdev | BL1 vs PRO/ |
| | range (median) | range (median) | range (median) | range (median) | BL2 vs PL |
| LB total count | 5.1 ± 1.9 | 6.7 ± 1.0 | 5.7 ± 1.4 | ND | 0.025/ |
| cultivation | 0-6.3 (5.9) | 4.9-8.6 (6.7) | 4.0-8.6 (5.3) | | |
| LB total count | 8.4 ± 0.1 | 8.4 ± 0.3 | 8.4 ± 0.3 | 8.3 ± 0.3 | 0.748/0.244 |
| FISH | 8.1-8.6 (8.4) | 7.9-9.1 (8.3) | 8.0-8.8 (8.3) | 7.6-9.0 (8.2) | |
| L. plantarum* | 0-5.3 (0) | 0-8.6 (5.9) | 0-4.3 (0) | ND | 0.006/ND |
| range/median | 3/12 | 10/12 § | 2/12 | | |
| prevalence | | | | | |

ND—not determined

The total count of lactobacilli and the prevalence of L. plantarum* increased (p=0.006) in faeces of volunteers.

TABLE 12
Prevalence of L. plantarum as species in fecal samples of volunteers at the
recruitment (BL1), after probiotic cheese consumption (PRO) and recovery
(BL 2)

| Group | BL 1 | PRO | BL 2 |
|---|---|---|---|
| Group 1 (n = 12) | 3/12 | 10/12 | 2/12 |
| Group 2 (n = 12) | 2/12 | 3/12 | 4/12 |

Increase of L. plantarum as species was detected (Fischer exact test, 3/12 vs 10/12, p=0.006) in the L. plantarum Tensia DSM 21380 group.

Thus, L. plantarum Tensia DSM 21380 affects positively human GIT microflora though increase of beneficial lactobacilli counts.

Surprisingly, by increasing the counts of facultative heterofermentative lactobacilli (PHEL; 4.7±1.3 vs. 5.7±1.3, p=0.029), the fermentation group, where L. plantarum Tensia DSM 21380 belongs, the increase of obligatively homofermentative lactobacilli (OHOL) count was detected (4.6±1.1 vs. 5.7±1.5, p=0.014). Thus, L. plantarum Tensia DSM 21380 helps significantly to stabilize GT lactoflora.

Fecal Lactobacilli Counts of Volunteers by Molecular Methods (Fluorescent In Situ hybridization, FISH)

Method. Fecal samples were diluted to 1/10 in PBS buffer. Microbial cells were fixed with 4% formaldehyde solution and kept at 4° C. FISH was carried out by method of Harmsen with the probe Lab 158 tagged with dye Cy 3. Tagged microbial cells were counted fluorescence microscopicy.

Results are presented in table 11 together with the results of bacteriological analyses. According to FISH, (the method that registers also dead cells) the lactobacilli counts remained practically unchanged.

The Lactobacilli DGGE Profile from Faeces of L. plantarum Tensia DSM 21380 Comprising Volunteers that Consumed Cheese.

Method. Subsequently, the PCR product was separated by DGGE electrophoresis in 30-60% acrylamide containing gel with Dcode™ System technique. (Bio-Rad, Hercules, Calif.). Gels were analyzed by BioNumerics 2.5 (Applied Maths, Belgium) software according to Peasoni correlation (Heilig H G, Zoetendal E G, Vaughan E E, Marteau P, Akkermans, A D L, de Vas W M. Molecular diversity of Lactobacillus ssp. and other Lactic acid bacteria in the human intestine as determined by specific amplification of 16S ribosomal DNA. Appl Envir Microbiol 2002; 68: 114-123).

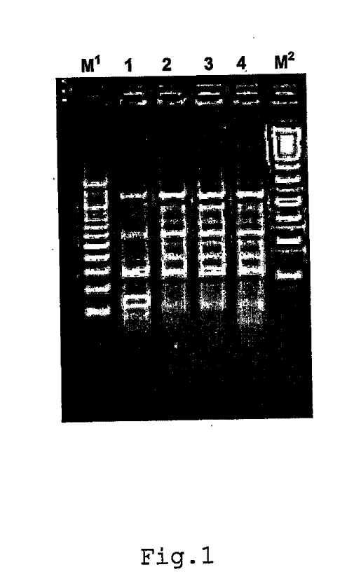

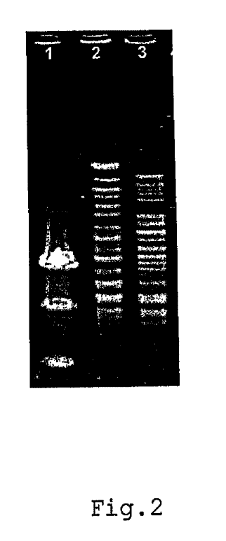

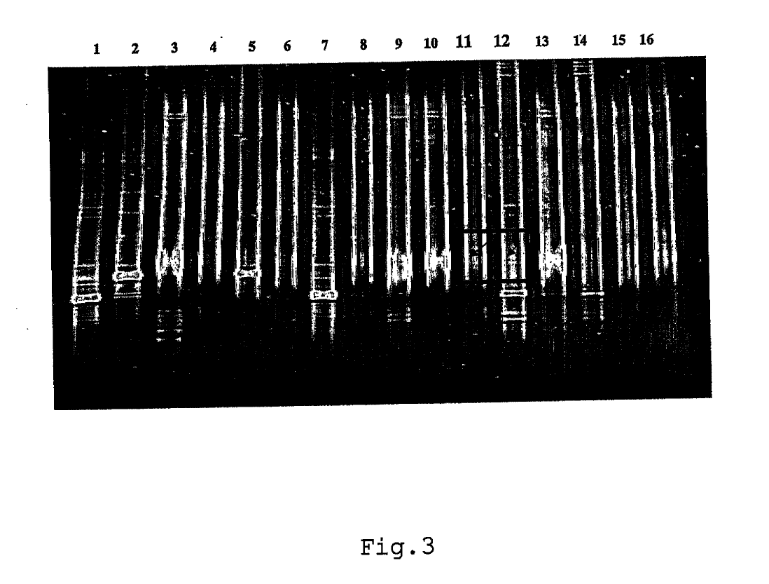

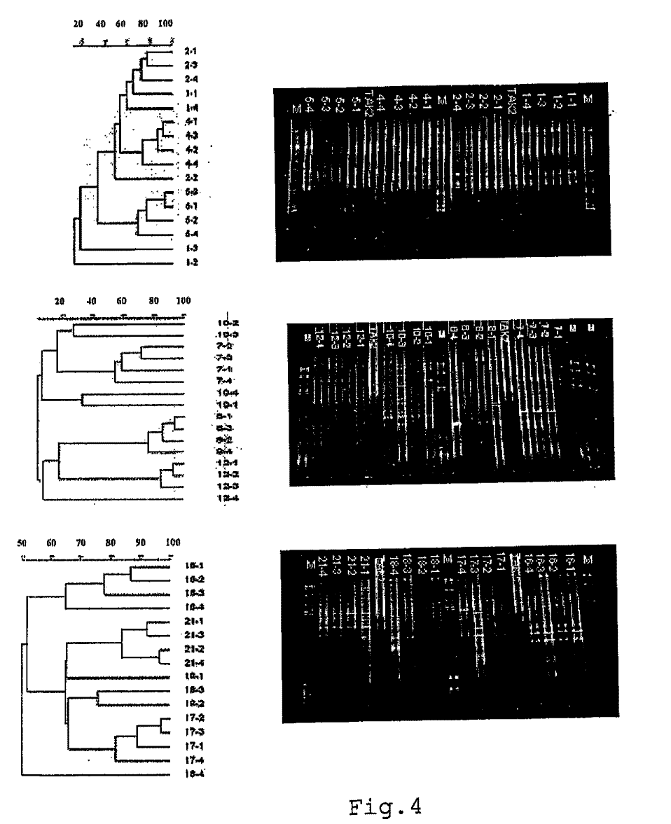

Gel photo (FIG. 4) indicates profile of dominant lactobacilli species in the subject's faeces according to 16S rDNA amplification. With the UMPAG software it was possible to analyze putative matrix coincidence between different gels, i.e. to detect significant difference (>20%) between different gels/different analysis.

TABLE 13
Changes in lactoflora in L. plantarum Tensia DSM 21380 group during
the clinical trial

| | | | | |
|---|---|---|---|---|
| Person | Day 0 | Day 21 | Day 35 | Day 56 |

1
2
4
5
7
8
10
12
16
17
18
21

Table 13 indicates, that in the L. plantarum Tensia DSM 21380 group the indigenous lactoflora pattern changed in 10 persons from 12 to the day 21 (p<0.05).

According results were obtained in L. plantarum colonization survey by bacteriological method. The changes remained stabile in 4 persons even after 2 weeks.

### The Polyamines and Biogenic Amines Content in the Urine of Volunteers

For the evaluation of biogenic amines before and after L. plantarum Tensia DSM 21380 comprising cheese consumption and at the stabilization period, the morning urine and gas chromatography method were used.

Method. Urine samples were derivatized with propylchlorophormate for GC analyze by modified method of Ugland (Ugland H G; Krough M, Rasmussen K E: Aqueous alkylchloroformate derivatization and solid-phase microextraction: determination of amphetamines in urine by capillary gas chromatography. J Chromatography B Biomed Sci Appl 1997; 701:29-38).

GC analysis were carried out by gas chromatograph HP 6890 Series GC System (Hewlett Packard, Avondale, Pa., USA), with capillar colonne HP-5 19091J-433 (30 m×0.25 mm; 0.25 μm) The column temperature program 150° C. 1 min, 20° C./min 280° C. for 5 min; and detector (FID) 250° C. The biogenic amines concentration was calculated according to nmol/mol creatinine.

TABLE 14
Polyamines in volunteers consuming L. plantarum Tensia DSM 21380 cheese assessed in
morning urine (nmol/mol creatinine)

| | Probiotic cheese with | | | | |
|---|---|---|---|---|---|
| | L. plantarum Tensia DSM | | | | |
| | 21380 additive | | Control cheese | | |
| | BL1 | PRO | BL2 | PL | P values |
| | mean ± stdev | mean ± stdev | mean ± stdev | mean ± stdev | paired t-test |
| | range (median) | range (median) | range (median) | range (median) | BL1 vs PRO/BL2 vs PL |
| Put | 0.110 ± 0.139 | 0.090 ± 0.120 | 0.073 ± 0.110 | 0.027 ± 0.024 | 0.496/0.275 |
| | 0-0.467 (0.046) | 0-0.396 (0.041) | 0-0.384 (0.037) | 0-0.055 (0.037) | |
| acPut | 0.607 ± 0.558 | 0.567 ± 0.431 | 0.817 ± 1.027 | 0.573 ± 0.391 | 0.510/0.677 |
| | 0-2.159 (0.478) | 0.191-1.758 (0.430) | 0.091-3.916 (0.550) | 0.182-1.403 (0.486) | |
| DAP | 0.148 ± 0.125 | 0.056 ± 0.084 | 0.090 ± 0.163 | 0.061 ± 0.099 | 0.016/0.844 |
| | 0-0.344 (0.159) | 0-0.205 (0) | 0-0.432 (0) | 0-0.246 (0) | |
| acSpd | 0.181 ± 0.137 | 0.227 ± 0.142 | 0.197 ± 0.148 | 0.258 ± 0.221 | 0.016/0.244 |
| | 0-0.428 (0.144) | 0.053-0.493 (0.182) | 0.039-0.495 [symbol] .195) | 0.041-0.686 (0.186) | |
| Cad | 0.012 ± 0.026 | 0.038 ± 0.062 | 0.041 ± 0.077 | 0.015 ± 0.037 | 0.125/0. |
| | 0-0.084 (0) | 0-0.156 (0) | 0-0.248 (0) | 0-0.123 (0) | |

BL1- baseline 1, at the recruitment,
PRO -after probiotic treatment,
BL2 - baseline 2, recovery
Put - putrescine,
acPut - N-acetylputrescine,
DAP - 1.3-diaminopropane,
acSpd - N8-acetylspermidine,
Cad - cadaverine [symbol]
indicates data missing or illegible when filed

Consumption of L. plantarum Tensia DSM 21380 comprising probiotic cheese increased the acetylated spermidine in urine of volunteers at the end of probiotic period indicating the improvement of the metabolism of polyamines in blood and tissues. DAP concentration in urine decreased.

### Correlations

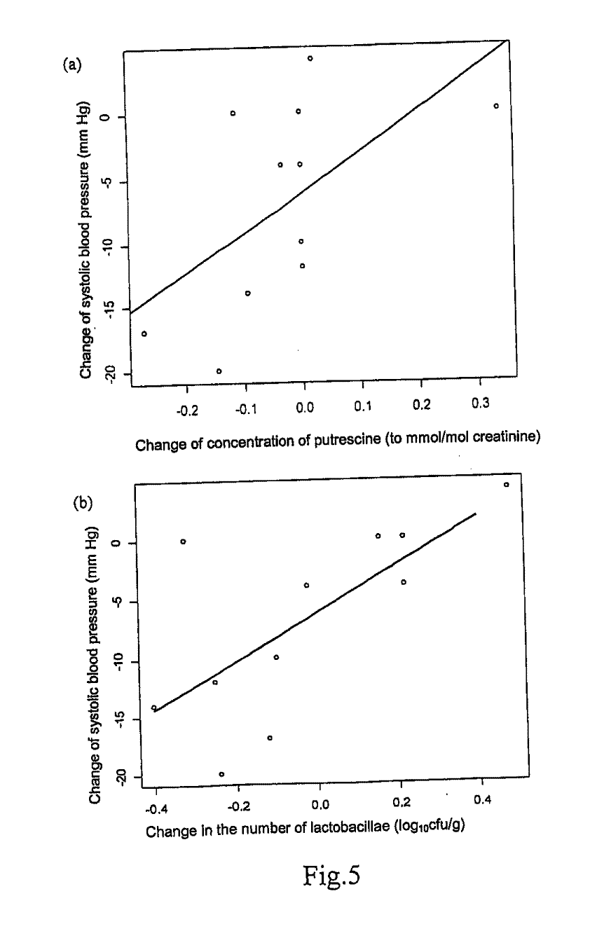

It was found that the reduction of systolic blood pressure was correlated to the increase of lactobacilli counts and the increase of putrescine in urine (FIG. 5).

### Safety

After cheese trial with volunteers, the values of systemic inflammation markers (U-CRP, ultrasensitive CRP, and leucocytes) were not changed and were within the normal range (Table 15). No change was detected also in the values of essential allergy marker IgE. The consumption of probiotic cheese did not cause changes in WBC counts (leucogram): the proportion of cells at the end of the trial remained unchanged in comparison with the recruitment values.

TABLE 15
Inflammation markers at the beginning and the end of the trial

| | Probiotic cheese with | | | | | |
|---|---|---|---|---|---|---|
| | L. plantarum Tensia | | | | | |
| | DSM 21380 | | Control cheese | | | Standard |
| | before | after | before | after | P values | values |
| U-CRP, mg/L | 1.1 ± 0.6 | 1.0 ± 0.3 | 1.4 ± 0.9 | 1.6 ± 1.3 | 0.3/0.9 | <5 mg/L |
| Leucocytes | 5.2 ± 0.8 | 5.6 ± 1.3 | 5.1 ± 1.1 | 5.5 ± 1.1 | 0.6/0.14 | 4-10 × 10⁹/L |
| total count × 10⁹/L | | | | | | |

Consumption of L. plantarum Tensia DSM 21380 comprising probiotic cheese did not cause abdominal discomfort (abdominal pain, flatulence, bloating) or the increase of the body mass index, glucose and lipid levels.

Thus in healthy subjects the consumption of Lactobacillus plantarum Tensia DSM 21380 comprising cheese does not cause systemic inflammation, allergic sensibilisation nor does it cause harm to essential organs.

### Example 4

### Effect of Lactobacillus plantarum DSM 21380 Comprising Cheese on Blood Indices of Elderly Healthy Individuals

During the clinical trial (randomized blinded cross-over placebo controlled) the safety and effect on blood indices of Lactobacillus plantarum Tensia DSM 21380 comprising cheese on elderly healthy volunteers was evaluated.

Persons and Methods. Participants were healthy volunteers, both male and female 21 persons (M/F 2/19; 61-84 years). For exclusion criteria diabetes, glucose and glycohemoglobin HbA1c from blood sera were detected.

Test cheeses comprised strain Lactobacillus plantarum Tensia DSM 21380 (viable counts of 2×10⁷ CFU/g cheese). Before consumption the test-cheese was incubated with Lactobacillus plantarum Tensia DSM 21380 for 30 days at 12° C. Regular Estonian cheese without additives served as a control. Trial was a randomized blinded cross-over placebo controlled trial. Trial started with 3-week consumption of test-cheese, followed by 2 week washout period, after which the control-cheese was consumed for 3 weeks. Dose 50 g/day.

TABLE 16
Clinical data of elderly healthy volunteers after consumption of probiotic L. plantarum
Tensia DSM 21380 comprising cheese

| | Probiotic cheese | | | | |
|---|---|---|---|---|---|
| | with L. plantarum | | | | |
| | Tensia DSM 21380 | | Control cheese | | |
| | | After | | After | |
| | Baseline | treatment | Baseline | treatment | P values |
| BMI (kg/m²) | 27.6 ± 4.1 | 27.5 ± 4.2 | 27.5 ± 4.0 | 27.6 ± 4.2 | 0.723/0.793 |
| Systolic | 138.1 ± 16.6 | 132.2 ± 16.2 | 138.7 ± 21.4 | 135.2 ± 21.1 | 0.038/0.185 |
| pressure | | | | | |
| (mmHg) | | | | | |
| Diastolic | 77.2 ± 7.7 | 73.1 ± 8.0 | 76.4 ± 8.9 | 74.8 ± 9.2 | 0.004/0.246 |
| pressure | | | | | |
| (mmHg) | | | | | |

Body mass index (BMI) (kg/m²): 19-25 kg/m² - normal, 26-30 kg/m² - overweight, over 30 - obesity.

The decrease of blood pressure (both systolic and diastolic) was detected on elderly subjects after the 3-week consumption of probiotic L. plantarum Tensia DSM 21380 (2×10⁷/g×50 g) comprising cheese. At the same time the consumption of cheese of relatively high fat content did not increase the body mass index of elderly volunteers (Table 16).

### Safety

The consumption of L. plantarum DSM 21380 comprising probiotic cheese by elderly did not cause abdominal discomfort (abdominal pain, flatulence, bloating).

After the cheese trial the values of systemic inflammation markers (U-CRP, and leucocytes) of the participants were not increased and were within the normal range (Table 17).

TABLE 17
The inflammation markers of blood of the elderly healthy
volunteers at the recruitment and at the end of the trial

| | Probiotic L. plantarum | | | | | |
|---|---|---|---|---|---|---|
| | Tensia DSM 21380 | | | | | |
| | comprising cheese | | Control cheese | | | Standard |
| | before | after | before | after | P values | values |
| U-CRP, mg/L | 0.13/0.81 | 0.13/0.81 | 0.13/0.8 | 0.13/0.81 | 0.13/0.81 | <5 mg/L |
| Leucocytes | 5.1 ± 1.3 | 4.9 ± 1.3 | 5.0 ± 1.0 | 5.0 ± 1.6 | 0.33/0.33 | 4-10 × 10⁹/L |
| total count × 10⁹/L | | | | | | |

No abnormalities were detected also in the values of essential allergy marker IgE or the kidney and liver markers (serum creatinine, albumine, alanine transaminase (ALAT), aspartate transaminase (ASAT)).

Comparing the total cholesterol levels of the treatment and placebo period, significant decrease was detected after the consumption of the L. plantarum DSM 21380 comprising cheese. The raise of the blood serum glucose content by 0.3 units after the probiotic cheese consumption was an undesirable effect, but in comparison with the placebo period the change was not statistically significant (Table 18) and remained within the normal range.

TABLE 18
Blood serum biochemical parameters of elderly healthy volunteers after consumption of
L. plantarum DSM 21380 comprising cheese.

| | | | | | P values |
|---|---|---|---|---|---|
| | Probiotic cheese | | Control cheese | | (BL1 vs BL2, |
| | BL1 | PRO | BL2 | PL | PRO vs PL) |
| Glycose, mmol/l | 5.1 ± 0.5 | 5.4 ± 0.5 | 5.3 ± 0.5 | 5.4 ± 0.4 | 0.036/0.144 |
| | | | | | (0.212, 0.757) |
| Total cholesterol, | 5.7 ± 0.8 | 5.6 ± 0.8 | 5.9 ± 0.9 | 5.7 ± 0.8 | 0.343/0.198 |
| mmol/l | | | | | (0.044, 0.087) |
| HDL-cholesterol, | 1.7 ± 0.4 | 1.6 ± 0.4 | 1.7 ± 0.4 | 1.7 ± 0.5 | 0.411/0.514 |
| mmol/l | | | | | (0.073, 0.118) |
| LDL-cholesterol, | 3.9 ± 0.8 | 3.8 ± 0.7 | 4.1 ± 0.9 | 3.8 ± 0.7 | 0.557/0.052 |
| mmol/l | | | | | (0.075, 0.9187) |
| Triglycerides, mmol/l | 1.1 ± 0.6 | 1.1 ± 0.6 | 1.2 ± 0.5 | 1.1 ± 0.5 | 0.380/0.394 |
| | | | | | (0.411, 0.097) |

Thus in elderly healthy individuals the consumption of Lactobacillus plantarum Tensia DSM 21380 comprising cheese does not cause systemic inflammation, allergic sensibilisation nor cause harm to essential organs.

1. An isolated probiotic microorganism strain Lactobacillus plantarum Tensia DSM 21380, that produces conjugated linoleic acid (CLA), hydrogen peroxide (H₂O₂) and nitric monooxide (NO).

2. The probiotic microorganism strain of claim 1 in freeze-dried form.

3. The isolated probiotic microorganism of claim 1 wherein said microorganism possesses antioxidative activity.

4. The isolated probiotic microorganism of claim 1 wherein said microorganism possesses plantaricin encoding genes plnE, plnF, plnJ and plnK.

5. The isolated probiotic microorganism strain of claim 1, wherein said microorganism is capable of reducing blood pressure.

6. The isolated probiotic microorganism strain of claim 1, wherein said microorganism possesses antimicrobial activity and is capable of suppressing contaminating microbes in a food product.

7. The isolated probiotic microorganism strain of claim 6, wherein said contaminating microbes are at least one of the group consisting of a non-starter lactobacilli, pathogens originated from raw milk, and a pathogen occasionally contaminating a food product after preparation.

8. The isolated probiotic microorganism strain of claim 1, wherein said microorganism is capable of achieving a dominant position among intestinal lactobacilli.

9. The isolated probiotic microorganism strain of claim 1, wherein said microorganism is capable of increasing polyamine metabolism in a human organism.

10. An antimicrobial and antihypertensive probiotic composition, comprising probiotic microorganism strain of claim 1.

11. The composition of claim 10, wherein said composition is selected from the group consisting a pharmaceutical composition and a food composition.

12. A food product comprising the probiotic microorganism strain of claim 1.

13. The food product of claim 12, wherein the food product is a dairy product.

14. The food product of claim 13, wherein the food product is a fermented milk product.

15. The food product of claim 14, wherein the food product is cheese.

16. A method for suppressing contaminating microbes in a food product comprising a step of adding to a food product the probiotic microorganism of claim 1.

17. The method of claim 16, wherein said microbes are at least one of the group consisting of a non-starter lactobacilli, pathogens originated from raw milk, and a pathogen occasionally contaminating a food product after preparation.

18. The method of claim 17, wherein the food product is a dairy product.

19. The method of claim 18, wherein the dairy product is a fermented milk product, cheese, cottage cheese, curds, yoghurt, ice-cream, butter, or spread cheese.

20. The method for the extension of the shelf life of a food product comprising a step of adding the probiotic microorganism of claim 1 to a food product.


SEQUENCE LISTING (10 sequences)

SEQ ID NO: 1 - DNA, 17 bp, Lactobacillus plantarum
  primer_bind (375855) (375871): Designation of the sequence E1F, location the sequence measured against the positive control Lactobacillus plantarum WCFS1
atcggcaggcccaacag

SEQ ID NO: 2 - DNA, 21 bp, Lactobacillus plantarum
  primer_bind (375954) (375934): Designation of the sequence E1R, location the sequence measured against the positive control Lactobacillus plantarum WCFS1
gcgtgaccgtgaattaaatgc

SEQ ID NO: 3 - DNA, 21 bp, Lactobacillus plantarum
  primer_bind (375837) (375857): Designation of the sequence F1F, location of the sequence measured against the positive control Lactobacillus plantarum WCFS1
gacagcgctaatgacccaatc

SEQ ID NO: 4 - DNA, 21 bp, Lactobacillus plantarum
  primer_bind (375954) (375934): Designation of the sequence F1R, location the sequence measured against the positive control Lactobacillus plantarum WCFS1
gcgtgaccgtgaattaaatgc

SEQ ID NO: 5 - DNA, 19 bp, Lactobacillus plantarum
  primer_bind (366953) (366971): Designation of the sequence J1F, location the sequence measured against the positive control Lactobacillus plantarum WCFS1
cccaacaaccccgttcaac

SEQ ID NO: 6 - DNA, 26 bp, Lactobacillus plantarum
  primer_bind (367060) (367035): Designation of the sequence J1R, location of the sequence measured against the positive control Lactobacillus plantarum WCFS1
tgctacagtaaagggagaggtatgac

SEQ ID NO: 7 - DNA, 27 bp, Lactobacillus plantarum
  primer_bind (366670) (366696): Designation of the sequence K2F, location the sequence measured against the positive control Lactobacillus plantarum WCFS1
acttattataatcccttgaaccaccaa

SEQ ID NO: 8 - DNA, 25 bp, Lactobacillus plantarum
  primer_bind (366796) (366772): Designation of the sequence K2R, location the sequence measured against the positive control Lactobacillus plantarum WCFS1
gctgacgctgaaaagaatatttctg

SEQ ID NO: 9 - DNA, 21 bp, Escherichia coli
  primer_bind (503858) (503872): Designation of the sequence 968-GC-f, location the sequence measured against the positive control Lactobacillus plantarum WCFS1
gggaacgcgaagaaccttagc

SEQ ID NO: 10 - DNA, 17 bp, Escherichia coli,
  primer_bind (504289) (504273): Designation of the sequence 1401-r location the sequence measured against the positive control Lactobacillus plantarum WCFS1
cggtgtgtacaagaccc